(19) United States
(12) Patent Application Publication
(10) Pub. No.: US 2024/0003921 A1
(43) Pub. Date: Jan. 4, 2024

(54) A LIPID BASED INDICATOR FOR CARDIOVASCULAR DISORDER

(71) Applicant: INDIAN INSTITUTE OF SCIENCE, Bangalore (IN)

(72) Inventors: Siva UMAPATHY, Bangalore (IN); Venkatesh KALIAPERUMAL, Bangalore (IN); Ria MUKHERJEE, Bangalore (IN)

(21) Appl. No.: 18/032,196

(22) PCT Filed: Dec. 15, 2021
(86) PCT No.: PCT/IB2021/061760

§ 371 Date: Apr. 17, 2023

(30) Foreign Application Priority Data
Oct. 16, 2020 (IN) 202041045171

(51) Int. Cl. G01N 33/92; G01N 21/65; G01N 21/3577

(52) U.S. Cl. CPC G01N 33/92; G01N 21/65; G01N 21/3577; G01N 2800/32

(57) ABSTRACT

The invention provides a method for rapid detection of the viability of microorganisms. The method includes capturing a first unique signature with respect to a first lipid component, capturing a second unique signature with respect to a second lipid component, transforming each of the first lipid signatures and second lipid signatures to obtain amplified signatures, comparing the amplified signatures to obtain a first ratio and repeating the abovementioned steps at least one more time to obtain a second ratio. The alteration in the first ratio and the second ratio provides the indication for cardiovascular disorder.

### FIELD OF INVENTION

The invention generally relates to the field of vibrational spectroscopy and particularly to a method for identification of a lipid based indicator for cardiovascular disorder using vibrational spectroscopy.

### BACKGROUND

Cardiovascular disorder poses a major health risk to human population. Thickening of the blood vessels or atherosclerosis due to lipid deposition leads to decrease in oxygen supply to the heart or brain resulting in heart attack and stroke respectively. Approaches for dealing with the disorder include but is not limited to identification of cardiovascular risk factors, application of medical technologies to treat acute coronary syndrome, and the development of interventions that reduces cardiovascular risk factors. Since cardiovascular disorder forms a significant problem for increasingly large population, detection of the disorder is of particular interest to researchers as well as general medical practitioners. In the recent times, early detection of the disorder through the identification of the risk factors has attracted the interest of the researchers and scholars. Patent CN101946009A discloses a method of diagnosis or prognosis of cardiovascular disease through biomarkers identification. Further, Patent US20040063216A1 mentions a method for rapid detection of biomarkers by subjecting the biological material to laser beam irradiation and obtaining spectral data for further comparisons. The challenges encountered when obtaining spectral data of biological material include but is not limited to baseline shifts, non-linear background and subtle spectral differences in health and disease states. This can lead to unwanted inaccuracies in the obtained spectra and inability to obtain quantitatively reproducible data. Hence, there is a pressing need to specifically address the aforementioned challenges and identify indicators that can be used for the rapid and accurate assessment of the cardiovascular risk.

### SUMMARY OF THE INVENTION

One aspect of the invention provides a method for identification of a lipid based indicator for cardiovascular disorder using vibrational spectroscopy. The method includes capturing a first unique signature with respect to a first lipid component. A second unique signature is captured with respect to a second lipid component. Each of the first lipid signature and second lipid signatures are then transformed to obtain amplified signatures of the first lipid component and the second lipid component. The amplified signatures are compared to obtain a first ratio. The abovementioned steps are then iterated at least one more time to obtain a second ratio. The alteration in the first ratio and the second ratio provides an indication for cardiovascular disorder.

### BRIEF DESCRIPTION OF DRAWINGS

So that the manner in which the recited features of the invention can be understood in detail, some of the embodiments are illustrated in the appended drawings. It is to be noted, however, that the appended drawings illustrate only typical embodiments of this invention and are therefore not to be considered limiting of its scope, for the invention may admit to other equally effective embodiments.

FIG. 1a shows low density lipoprotein (LDL) carotenoid to total lipid ratio in people with high risk for cardiovascular disorder in comparison with controls, according to an example of the invention.

FIG. 1b shows LDL phospholipid to triglyceride ratio in people with high risk for cardiovascular disorder in comparison with controls, according to an example of the invention.

### DETAILED DESCRIPTION OF THE INVENTION

Various embodiments of the invention provide a method for obtaining a lipid based indicator for cardiovascular disorder using vibrational spectroscopy.

The method includes capturing a first unique signature with respect to a first lipid component, capturing a second unique signature with respect to a second lipid component, transforming each of the first lipid signatures and second lipid signatures to obtain amplified signatures, comparing the amplified signatures to obtain a first ratio and repeating the abovementioned steps at least one more time to obtain a second ratio. The alteration in the first ratio and the second ratio provides an indication for cardiovascular disorder. The method described herein, briefly shall be discussed in detail, herein below.

The method for obtaining lipid based indicator for cardiovascular disorder is based on vibrational spectroscopy. Vibrational spectroscopy measures the spectra of microscopic samples. The technique captures molecular bond specific vibrations originating from the biochemical constituents. Examples of vibrational spectroscopy include but are not limited to Raman Spectroscopy and Infrared Spectroscopy.

First step of obtaining lipid based indicator for cardiovascular disorder using vibrational spectroscopy includes capturing a first unique signature with respect to a first lipid component. Second step is capturing a second unique signature with respect to a second lipid component. Examples of lipid component includes but is not limited phospholipids, carotenoids, total lipid of low density lipoproteins, cholesterol, triglycerides, and total lipid of high density lipoproteins.

According to an embodiment of the invention, the lipid component is sourced from mammalian source. Preferably, the lipid component is pre-extracted fluid. Examples of pre-extracted fluid include but are not limited to blood, cerebrospinal fluid.

According to another embodiment of the invention, the lipid component is from the same source or distinct source.

In one embodiment of the invention, signatures are obtained through Infra-red spectroscopy. Lipid component samples are mixed with Potassium Bromide KBr to prepare KBr pellette. Infra-red signatures are collected in transmission mode. The wave number of the electromagnetic radiation for obtaining Infrared signature is in the range of 400 cm⁻¹ to 4000 cm⁻¹. Spectra resolution is around 4 cm⁻¹.

In another embodiment of the invention, signatures are obtained through Raman spectroscopy. Lipid component samples are drop-casted on aluminium foil and signatures are recorded. A objective is used. The wavelength of the electromagnetic radiation for obtaining Raman signature is in the range of 442 nm to 830 nm. In one example of the invention, Raman signatures are collected using 514 nm laser using 2400 lines/mm grating. Signatures are collected using Wire 4.1 software. Spectral second derivative and area under curve is calculated using MATLAB. Multivariate analysis (MCR-ALS) is performed with CAMO Unscrambler-X software.

The signatures are then further subjected to transformation steps. The steps include eliminating at least one baseline shifts and non-linear background of the signatures, dimensionally reducing the base line shifted signatures and resolving the dimensionally reduced signatures to obtain amplified signatures. Subsequent to transformation, amplified signatures are compared to obtain a first ratio. The abovementioned steps are performed at least one more time to obtain a second ratio. Further, the obtained ratios are studied to check for any alternations. The alteration in the first ratio and the second ratio provides the indication for cardiovascular disorder.

The indicators in the present invention are known to show changes on the onset of the cardiovascular disorder. Examples of indicators include but are not limited to LDL-carotenoid to LDL-total lipid ratio and LDL-Phospholipid to LDL-Triglyceride ratio. In one example of the invention, ratio of LDL-Carotenoid to LDL-Total lipid ratio indicates high risk for the onset of the cardiovascular disease.

### Example 1

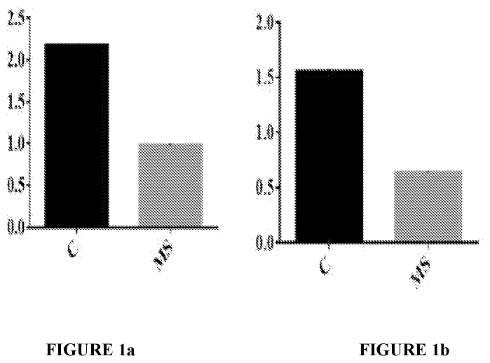

In one example, Lipid Raman signatures are pre-processed using a series of steps so that peaks resolve better. The pre-processing steps include smoothing, derivatization, distinct spectral peak identification and calculation of area under each peak. The captured signatures are subjected to multivariate curve resolution to identify major chemical constituents. Many of the resolved peaks in the second derivative signatures have predefined assignments to specific chemical bonds. There is a sharp cholesterol peak at 700 cm⁻¹. There are other sharp features from carotenoids and CH2 bending mode at 1440 cm⁻¹. The trans C═C band at 1672 cm⁻¹ and cis C═C band at 1659 cm⁻¹ are clearly resolved. The prominent peaks at 1552, 1157 and 1004 cm⁻¹ are observed, which belongs to the carotenoids while the peaks at 1658, 1440 and 700 cm⁻¹ are from the total lipids of Low Density Lipoprotein. FIG. 1a shows Low Density Lipoprotein Carotenoid to total lipid ratio in people with high risk for cardiovascular disorder in comparison with controls obtained by dividing concentrations of two components in two respective components MCR analysis. In the Raman spectroscopic study, carotenoids decreases in a person with high risk for cardiovascular disorder. MCR analysis suggests out-of-phase relation between carotenoids and total lipid and the concentration ratios of these two-component emerged as an indicator for cardiovascular disorder. Similarly, three component MCR analysis has identified components corresponding to cholesterol, phospholipids and triglycerides. The phospholipid-triglyceride ratio determined from these components is shown in FIG. 1b. It shows decreased LDL Phospholipid to triglyceride ratio in people with high risk for cardiovascular disorder. In three component MCR result, contribution from the changes in triglyceride and phospholipid ratio becomes important for risk assessment.

LDL is implicated in atherosclerotic plaque formation and blockage of blood vessels. But blood LDL cholesterol level is not a useful indicator of the atherosclerotic risk. The relative lipid component constituents differentiate high risk individuals from low risk healthy subjects. In addition to risk assessment, the quantitative biochemical markers described herein will serve as a useful indicator in evaluating treatment response and effect of lifestyle modifications. While other commonly used surrogate lipid markers like Serum Triglycerides and HDL cholesterol do not address the LDL pathology, the present invention focuses on LDL which is responsible for atherosclerosis.

The foregoing description of the invention has been set merely to illustrate the invention and is not intended to be limiting. Since modifications of the disclosed embodiments incorporating the spirit and substance of the invention may occur to person skilled in the art, the invention should be construed to include everything within the scope of the appended claims and equivalents thereof.

We claim:

1. A method of obtaining a lipid based indicator for cardiovascular disorder using vibrational spectroscopy, the method comprising:
capturing a first unique signature with respect to a first lipid component;
capturing a second unique signature with respect to a second lipid component;
transforming each of the first lipid signatures and second lipid signatures to obtain amplified signatures;
comparing the amplified signatures to obtain a first ratio; and
repeating the abovementioned steps at least one more time to obtain a second ratio.
Wherein, alteration in the first ratio and the second ratio provides the indication for cardiovascular disorder.

2. The method as claimed in claim 1, wherein the captured unique signature is either a Raman signature or an Infrared signature.

3. The method as claimed in claim 1, wherein the wavelength of the electromagnetic radiation for obtaining Raman signature is in the range of 442 nm to 830 nm.

4. The method as claimed in claim 1, wherein the wave number of the electromagnetic radiation for obtaining Infrared signature is in the range of 400 cm⁻¹ to 4000 cm⁻¹.

5. The method as claimed in claim 1, wherein the transformation of signatures includes steps:
eliminating at least one baseline shifts of the signatures;
dimensionally reducing the base line shifted signatures; and
resolving the dimensionally reduced signatures to obtain amplified signatures.

6. The method as claimed in claim 1, wherein lipid component is at least one of phospholipids, carotenoids, total lipid of low density lipoproteins, and total lipid of high density lipoproteins.

7. The method as claimed in claim 1, wherein the first lipid component and the second lipid component are identical or distinct.
